(19) United States
(12) Patent Application Publication
(10) Pub. No.: US 2010/0114097 A1
(43) Pub. Date: May 6, 2010

(54) Implant Devices Constructed with Metallic and Polymeric Components

(75) Inventors: Mark Siravo, Norristown, PA (US); Glen Pierson, Glenmoore, PA (US)

Correspondence Address: Fay Kaplun & Marcin, LLP, 150 Broadway, suite 702, New York, NY, 10038, US

(73) Assignee: Synthes USA, LLC

(21) Appl. No.: 12/524,427

(22) PCT Filed: Apr. 18, 2008
(86) PCT No.: PCT/US08/60810

§ 371 Date: Jul. 24, 2009

Related U.S. Application Data
(60) Provisional application No. 60/914,482, filed on Apr. 27, 2007.

(51) Int. Cl. A61B 17/56; A61B 17/80

(52) U.S. Cl. 606/62; 606/297

(57) ABSTRACT

A device for treating bone comprises a rigid body including a polymeric material extending over at least a target portion thereof. The device further comprises a locking element extending into the bone and attached to the device by forming a permanent bond therebetween by melting a portion of an outer surface of the locking element and the polymeric material.

### BACKGROUND

Various implants are used in the orthopedic field to stabilize portions of bone after a fracture, following an osteotomy procedure, or prophylactically to prevent fractures of bone weakened due to tumor, disease, etc. These implants include, for example, fixation plates and intramedullary nails. Such plates and nails typically are constructed of either biocompatible metallic materials or biocompatible polymeric materials. Purely metallic devices constructed, for example, of titanium alloy, have the advantage of increased strength but require mechanical fixation means such as screws while polymeric devices are sometimes difficult to clearly visualize under fluoroscopy.

### SUMMARY OF THE INVENTION

A device according to the present invention is directed to treating a bone, the device comprising rigid body including a polymeric material extending over at least a target portion thereof. The device further comprises a locking element extending into the bone and attached to the device by forming a permanent bond therebetween by melting a portion of an outer surface of the locking element and the polymeric material.

### BRIEF DESCRIPTION OF THE DRAWINGS

FIG. 1 shows a view of an exemplary fixation apparatus according to the present invention inserted within a bone;

FIG. 2 shows a perspective view of an intramedullary nail of the apparatus of FIG. 1;

FIG. 3 shows a cross-sectional view of a distal tip of the intramedullary nail of FIG. 2;

FIG. 4 shows a perspective view of a locking element of the apparatus of FIG. 1;

FIG. 5 shows a first perspective view of the fixation apparatus of FIG. 1 partially inserted into the bone;

FIG. 6 shows a second perspective view of the fixation apparatus of FIG. 1 partially inserted into the bone and rotated about an axis of the bone relative to FIG. 5;

FIG. 7 shows a perspective view of an end cap according to the present invention;

FIG. 8 shows a cross-sectional view of a bore for receiving the end cap of FIG. 7;

FIG. 9 shows a cross-sectional view of a bone plate according to another exemplary embodiment of the invention;

FIG. 10 shows a cross-sectional view of a further embodiment of a bone plate according to the invention;

FIG. 11 shows a cross-sectional view of a still further embodiment of a bone plate according to the invention;

FIG. 12 shows a cross-sectional view of an additional embodiment of a bone plate according to the invention;

FIG. 13 shows a cross-sectional view of an additional embodiment of a bone plate according to the invention; and

FIG. 14 shows a perspective view of the bone plate of FIG. 13.

### DETAILED DESCRIPTION

The present invention is directed to devices for stabilizing portions of bone which may be employed either after a fracture or prophylactically to prevent fractures of weakened portions of bone (i.e., due to tumor or disease). A device according to the present invention comprises an implantable device (e.g., an intramedullary or extramedullary nail, bone plate, etc.) including both metallic and polymeric components and adapted to fix portions of bone in a living body. The present invention also teaches locking elements adapted to lock the device to the bone by passing through holes in the device into the bone. Specifically, a device according to the present invention is placed within or on a bone according to methods known in the art and coupled to the bone via fixation elements inserted either through the device into the bone or through the bone into the device. A core of the device is formed of a material with a stiffness greater than that of the polymeric portion. Specifically, the core may be metallic, carbon fiber or other polymeric material with substantially rigid properties designed to withstand pressures exerted theretagainst during insertion and retention in the bone. The fixation elements may then be permanently secured to the device (e.g., via adhesive, ultrasonic heating, etc.). Specifically, energy (e.g., heat, ultrasonic vibration) may be applied to a polymeric material of the locking element to permanently bond a polymeric portion of the device thereto. It is noted that although the embodiments of the present invention are described herein with respect to specific procedures and specific portions of the anatomy, they are not intended to limit the scope of the present invention, which may be used in any of a number of procedures such as, for example, treatment of pediatric fractures of long bones.

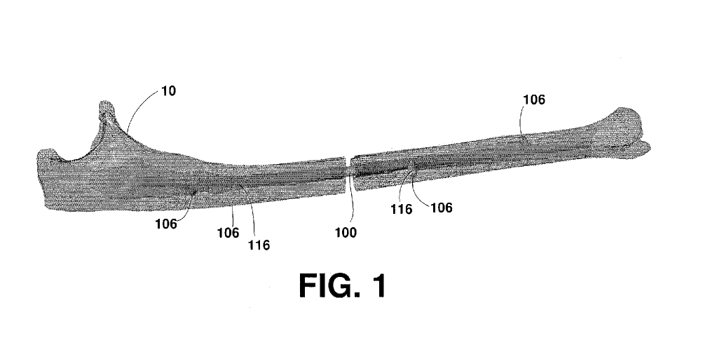

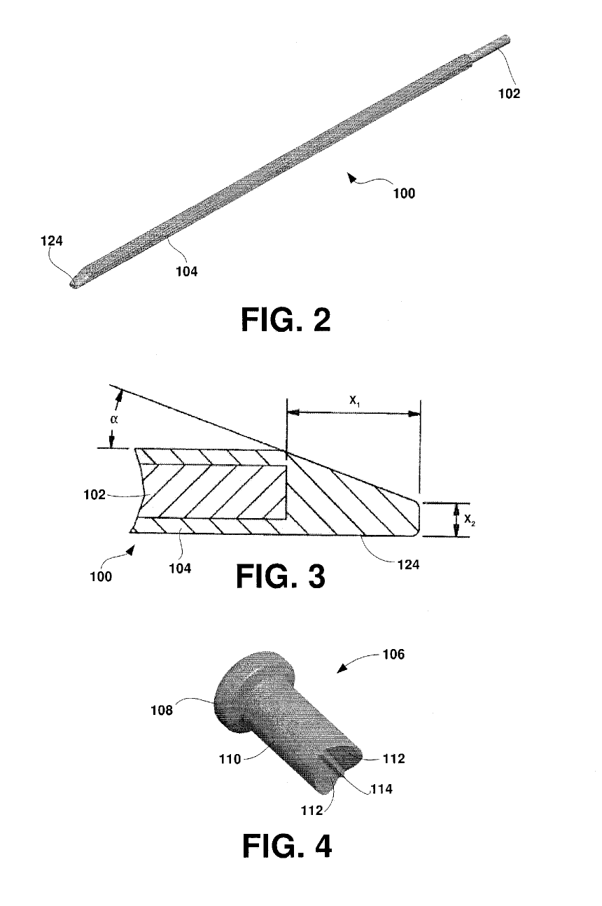

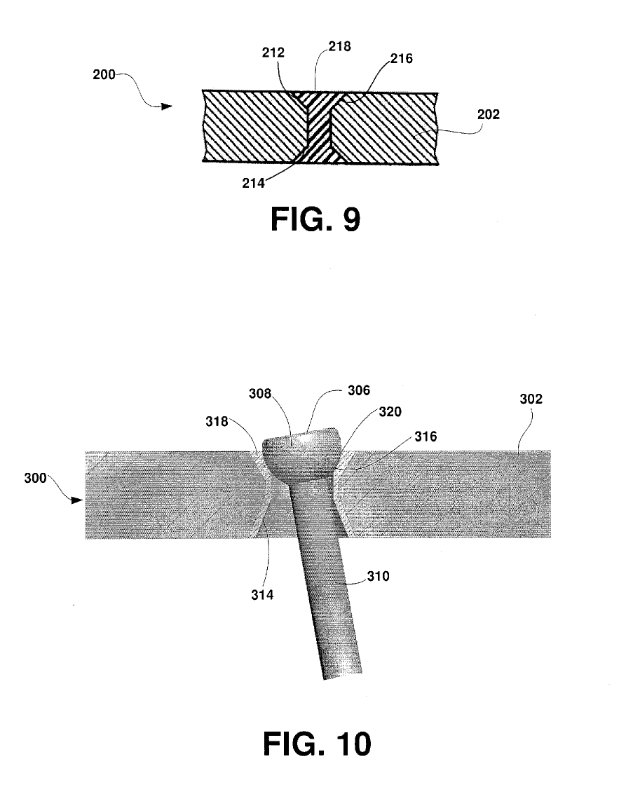

As shown in FIGS. 1-4, an intramedullary nail 100 according to a first embodiment of the invention is sized and shaped to be received within the medullary cavity of a bone 10 (e.g., the ulna). As would be understood by those skilled in the art, dimensions of the intramedullary nail 100 may be modified to conform to the dimensions of any long bone in the body (e.g., the forearm, the fibula, the clavicle, etc.). The intramedullary nail 100 comprises a core 102 comprised of any biocompatible metal such as, for example, a titanium alloy. It is noted that, although exemplary embodiments of the intramedullary nail 100 are described with a core 102, any material of a comparable rigidity may be employed without deviating from the scope of the present invention. For example, a metallic alloy, carbon fiber or another polymeric material may form the core 102. The core 102 is formed as an elongated substantially cylindrical core extending along substantially the entire longitudinal length of the intramedullary nail 100 and providing structural rigidity needed to stabilize a bone 10 which has been weakened or which includes a fracture such as the mid-shaft ulna fracture shown in FIG. 1. Those skilled in the art will understand that the nail 100 will not likely extend along a straight line and that, therefore, the term cylindrical is only a loose approximation for the shape of the core 102. More specifically, although a cross-section of the core 102 in a plane substantially perpendicular to a longitudinal axis of the nail 100 may be substantially circular, the true shape of the core 102 will be formed substantially as a series of circular sections extending along the curved path of the longitudinal axis of the nail 100. Alternatively, the core 102 may be substantially elliptical or otherwise non-circular with a similarly complex shape defined by a series of these cross-sectional shapes arranged along the curved path of the longitudinal axis of the nail 100. In a further embodiment of the invention, the shape of the core 102 may be specifically formed to match the anatomy of a bone into which it is to be inserted. Specifically, a proximal end thereof may be flared to fill a metaphyseal area, as those skilled in the art will understand. Alternatively, the core 102 and the intramedullary nail 100 may be formed with a non-circular cross-section to improve bony purchase thereof. For example, the cross-section may be formed with a star-shaped cross-section. Furthermore, the cross-section may be rectangular, as will be described in greater detail below with respect to the bone plates of FIGS. 9-12.

When deployed in a medullary canal of a target bone, the core 102 further serves as a visual indicator of the location of the intramedullary nail 100 under fluoroscopy providing a clearer image than non-metallic portions of the nail 100. Accordingly, fluoroscopy may be used to guide the intramedullary nail 100 into the bone 10. Furthermore, the core 102 provides a substantial coupling for any known instrument (not shown) for inserting and/or removing the intramedullary nail 100 to or from the bone 10. Specifically, by engaging the rigid core 102, such an implantation/explantation instrument can exert the required axial and/or torsional forces to the nail 102 without exceeding the strength of the nail 100.

A non-metallic casing 104 surrounds at least a portion of the core 102. As would be understood by those skilled in the art, the casing 104 may be formed as a polymeric shroud, covering or coating extending over at least a portion of the intramedullary nail 100 formed of a biocompatible material such as, for example, polyetheretherketone (PEEK), polylactide or UHMWPE. However, those skilled in the art will understand that the casing 104 is required only in areas to which it is desired to permanently bond a locking element 106. For example, it may be desirable to form the casing only over target areas to be contacted by the locking elements 106 while in other areas, the core 102 forms an outer surface of the nail 100.

In a preferred embodiment, as shown in FIGS. 1-3, the casing 104 covers the entire length of the core 102 and is permanently secured thereto. The casing 104 may, for example, be insert molded onto the core 102 or formed via an extrusion process, as those skilled in the art will understand. Alternatively, the casing 104 may be heat sealed to the core 102. The casing 104 preferably extends distally past a distal end of the core 102 to form a non-metallic distal tip 124, as shown in FIG. 1B. Specifically, the casing 104 extends past the core 102 by a distance X₁, preferably assuming a tapered shape to facilitate insertion of the nail 100 into the medullary canal. In a preferred embodiment, the casing 104 tapers at an angle α of approximately between 10° and 30° and, more preferably, approximately 20°. It is further submitted that the value of X₁ and α are directly related to one another to prevent the tapered portion from exceeding a minimum thickness X₂. Furthermore, it is noted that the values for X₁ and X₂ may vary with respect to the anatomy of the bone 10. An intramedullary nail according to an alternate embodiment of the present invention (not shown) may be formed with a core 102 that extends distally past the casing 104.

The casing 104 is adapted to accept at least one polymeric locking element 106, as shown in FIG. 4, to retain the intramedullary nail 100 in the bone 10. In use, the locking element 106 is permanently bonded or welded to the casing 104. The locking element 106 may also be formed of any suitable biocompatible polymeric material such as, for example polyetheretherketone (PEEK). The locking element 106 can be constructed solely from the polymeric material or, alternatively, may have a substrate of another material (i.e., metal, etc.) encased in the polymeric material. For example, the locking element 106 may include a metal core to provide structural rigidity thereto and to aid in location thereof using fluoroscopy in a manner similar to that described above for the nail 100. As would be understood by those skilled in the art, the locking element 106 may be formed as a locking tack with a head 108 having a diameter greater than that of a shaft 110 thereof. A distal end of the locking element 106 comprises two faces 112 angled to extend proximally from outer, distal-most ends toward a centrally located abutment 114. The faces 112 and the abutment 114 increase a surface area of the locking element 106 engaging a surface of the casing 104 of the nail 100 to enhance the bonding therebetween.

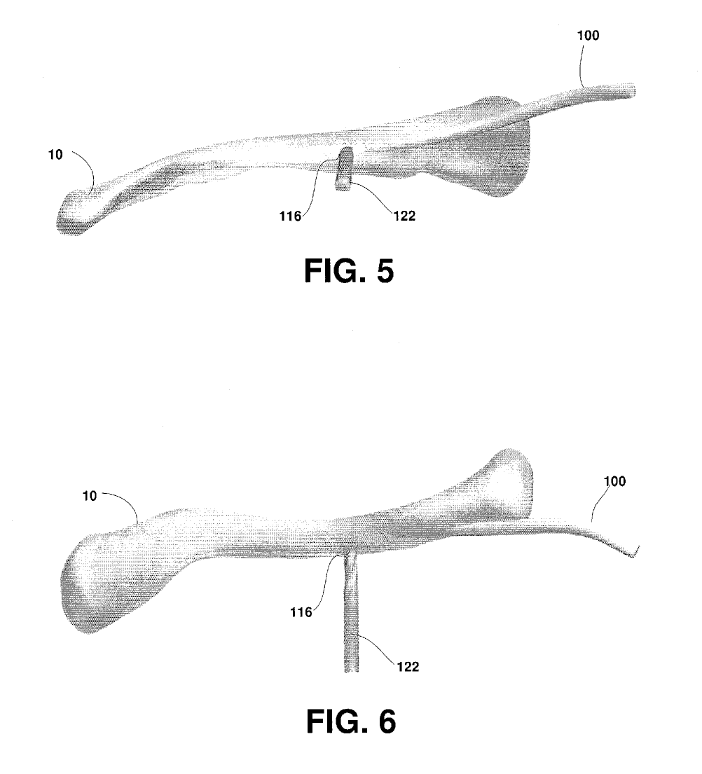

An exemplary method of use of the intramedullary nail 100 comprises inserting the intramedullary nail 100 into a medullary cavity of a designated long bone in the same manner as a conventional intramedullary nails. As shown in FIGS. 5 and 6, as the nail 100 is moved further into the medullary canal, its position is monitored (e.g. through fluoroscopic observation of the core 102) and, as the distal tip 124 nears a location at which it is desired to insert a locking element 106 (i.e., when a distal end of the core 102 has reached the location), the user may use the fluoroscopic image of the core 102 to ensure that a drill bit 122 of a drill (not shown) is aimed directly toward a portion of the nail 100 to which the locking element 106 is to be bonded. The drill is then operated to form a hole 116 through which the locking element 106 is to be inserted. As would be understood by those skilled in the art, designated hole locations may be calculated during preoperative planning and distributed along the length of the bone to provide the desired locking force holding the intramedullary nail 100 in a desired position within the medullary canal. Each of the holes 116 is drilled just before the intramedullary nail 100 passes the hole location. In this manner, a tip of the intramedullary nail 100 is used as a reference to ensure that the locking element 106 is coaxial with the intramedullary nail to ensure proper bonding while, at the same time, avoiding any potential damage to the casing 104 by the drill.

When all of the holes 116 have been drilled at the desired locations and the nail 100 has been inserted into the medullary canal to the desired position therein, a locking element 106 is inserted into one of the holes 116 until the angular faces 112 and the abutment 114 of the locking element 106 contact the casing 104 of the intramedullary nail 100. Application of pressure to the head 108 forces the locking element 106 against the casing and a source of energy (e.g., ultrasound vibration from an ultrasonic generator) is applied to the head 108 generating heat between the locking element 106 and the casing 104 and melting the polymeric materials thereof. These molten polymeric materials bond to one another, as those skilled in the art will understand to form a permanent connection between the locking element 106 and the casing 104. This process is then repeated to bond a locking element 106 to the casing via each of the holes 116. For example, a plurality of locking elements 106 may be disposed along all or a portion of the length of the nail 100 and at any desired angular orientations with respect to a longitudinal axis of the nail 100. Once bonded to the bone 10, any outlying portion of the head 108 is cut flush with the outer cortex of the bone so that no portion of the locking element 106 projects out of the bone 10. In this manner, the present invention offers substantially unlimited locking options for the intramedullary nail 100 (i.e., locking elements 106 may be placed at any desired locations), wherein any plurality of locking elements 106 may be employed depending on the requirements for a particular procedure.

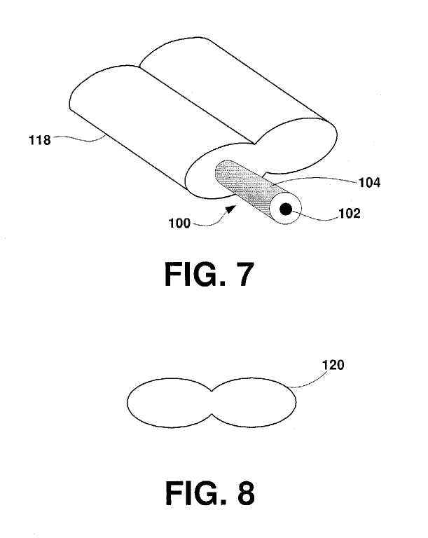

The intramedullary nail 100 may also be provided with an optional end cap to provide an additional means for preventing rotation thereof. As shown in FIGS. 7-8, an end cap 118 may provided over one or both ends of the intramedullary nail 100. An exemplary end cap 118 according to the present invention is non-circular in shape and is formed either of a biocompatible polymer known in the art or as a combination of a metal and a polymer material as disclosed earlier in regard to the nail 100 and the locking elements 106. FIG. 7 shows an end cap 118 in the shape of a figure eight, with two curved elements joined together. It is noted, however, that any non-circular shape is permissible, including, but not limited to, oval, rectangular, triangular, etc. After insertion of the intramedullary nail 100 into the medullary canal, an end cap 118 may be attached to the proximal end thereof. Specifically, an opening 120 is drilled into the end of the long bone, as shown in FIG. 8 just prior to the insertion of the intramedullary nail 100, providing the added benefit of easing the insertion of the intramedullary nail 100 into the bone. The depth and width of the opening 120 may be sized to match up with the dimensions of the end cap. Once inserted, the polymeric material of the end cap 118 can be bonded to the casing 104 via the application of heat thereto, as discussed with respect to FIGS. 1-6.

As shown in FIG. 9, an intramedullary nail 200 according to a further embodiment of the invention includes one or more holes 212 each for engaging a corresponding locking element 106. Specifically, the hole 212 of the nail 200 includes a polymeric insert 218 therein obviating the need for a casing 104. Accordingly, the intramedullary nail 200 may be formed entirely of a biocompatible metallic material with polymeric inserts 218 in the holes 212 thereof so that the polymeric inserts 218 may be employed to permanently bond the locking elements 106 to the nail 200 at the respective holes 212. That is, as the locking elements 106 may be permanently bonded to the inserts 218, they need not be bonded to a polymeric casing of the nail 200. However, as would be understood by those skilled in the art such a coating may be included if desired for any reason. The metallic portion 202 of the nail 200 may be formed of a material similar to that of the core 102 of the nail 100 described above in regard to FIGS. 1-3. The holes 212 are preferably formed in an hourglass shape with flared ends defined by angled faces 214, 216 at either end thereof as disclosed, for example, in International Application No. WO2004/110291 entitled “Surgical Nail” filed on Jun. 12, 2003 to Schlienger et al., the entire contents of which are incorporated herein by reference. This shape aids in maintaining the insert 218 constructed, for example, of a polymeric material suitable for bonding to a locking element 106 as described above, within the locking hole 212 even when subjected to forces along the axis of the hole 212 (e.g., by a locking element 106 inserted therethrough). The polymeric insert 218 preferably completely fills the void of the transverse locking hole 218. In an alternate embodiment, the polymeric inserts 218 may be formed as coatings covering at least a portion or preferably the entire surfaces of the angled faces 214, 216 and may, optionally extend out of the hole 212 along a portion of an outer surface of the nail 200. That is, the polymeric inserts 218 may be formed to be solid or alternatively may include a bore formed therethrough (not shown), the bore being longitudinally aligned with a longitudinal axis of the transverse locking hole 212 to receive a locking element 106 therethrough.

The polymeric inserts 218 are adapted to accept polymeric locking elements 106 that may be bonded or welded thereto to in the same manner described above in regard to the bonding between the casing 104 and the locking elements 106. Accordingly, once an intramedullary nail 200 has been implanted within a bone (not shown) in the same manner described above in regard to the nail 100, locking elements 106 may be fitted through preformed holes in the bore, as described earlier, so that angled faces 112 and abutment 114 lie in contact with the polymeric inserts 218. A permanent bond is then formed by causing a heating therebetween, as also disclosed earlier with respect to the embodiment of FIGS. 1-6.

As shown in FIG. 10, an exemplary bone fixation apparatus according to the present invention may also be formed as a bone fixation plate 300 comprising at least one locking element receiving aperture 320 therein. A wall of the aperture 320 is formed with a polymeric bushing 318 pre-installed and permanently bonded to the plate 300. As would be understood by those skilled in the art, the plate 300 may be constructed from any suitable material such as, for example, stainless steel, a titanium alloy, or a rigid core with a polymeric casing as described above in regard to the nail 100. The plate 300 may be further be constructed in any known fashion including apertures 320 for receiving any bone fixation elements (e.g., bone screws, pins, etc.) in the manner, for example, of any of the plates disclosed in U.S. Pat. No. 5,976,141 entitled “Threaded Insert for Bone Plate Screw Hole” filed on Feb. 23, 1995 to Haag et al., the entire contents of which are incorporated herein by reference. In an exemplary embodiment of the present invention, as shown in FIG. 10, the polymeric bushing 318, constructed from any of the materials described above, may include a bore extending therethrough for receiving the locking element 306 in the same manner described above. The aperture 320 receiving the polymeric bushing 318 is formed in a substantially hourglass shape to increase a surface bonding area with the polymeric bushing 318, thus ensuring a rigid bond therebetween. A proximal portion 316 of the polymeric bushing 318 is formed in a substantially semi-spherical shape, transitioning to a outwardly tapered shape at a distal portion 314 thereof. Furthermore, the polymeric bushing 318 may be formed with a greater diameter on a proximal side thereof, the diameter tapering to a reduced diameter at a central portion.

The semi-spherical shape of the proximal portion 316 of the polymeric bushing 318 is adapted to receive a locking element 306 so that a curvature of a head 308 of the locking element 306 substantially matches that of the proximal portion 316 allowing the locking element 306 to be angled as desired with respect to the plate 300. At least a portion of the locking element 306 is provided with a polymeric coating for bonding with the polymeric bushing 318. In the exemplary embodiment shown, only the head 308 of the locking element 306 is coated with a polymeric material while a shaft 310 thereof is metallic with no coating provided thereover. It is noted, however, that any or all portions of the locking element 306 may be provided with a polymeric coating without deviating form the scope of the present invention. In the same manner as the locking elements 106 described above, when the locking element is supplied with energy (e.g., ultrasound vibration) an outer portion of the polymeric bushing 318 is permanently bonded to the locking element 306.

In use, the polymeric bushing 318 is pre-molded into corresponding apertures 320 of the plate 300 and permanently bonded thereto the plate 300 in any known manner as described above in regard to the bonding of the locking elements 106 and the nail 100. The plate 300 which may, for example, be formed to conform to a contour of a target portion of bone to be treated, is placed over the target portion of bone and bores are drilled into the bone to receive one or more locking elements 306. A locking element 306 is then inserted through the aperture 320 in the plate 300 into a corresponding bore by being screwed or otherwise forced past the polymeric bushing 318. This is repeated for each locking element 306 to be inserted through the plate 300 into the bone. A permanent bond is then formed therebetween via application of energy (e.g., ultrasonic vibration) as discussed earlier. Of course, those skilled in the art will understand that, in any application, a plate 300 may receive one or more conventional fixation elements (e.g., bone screws or pins) through apertures formed in any known manner along with the one or more locking elements 306 which are permanently bonded to the inserts 318 by application of energy (e.g., ultrasound vibrations produced by an ultrasonic generator) as disclosed earlier.

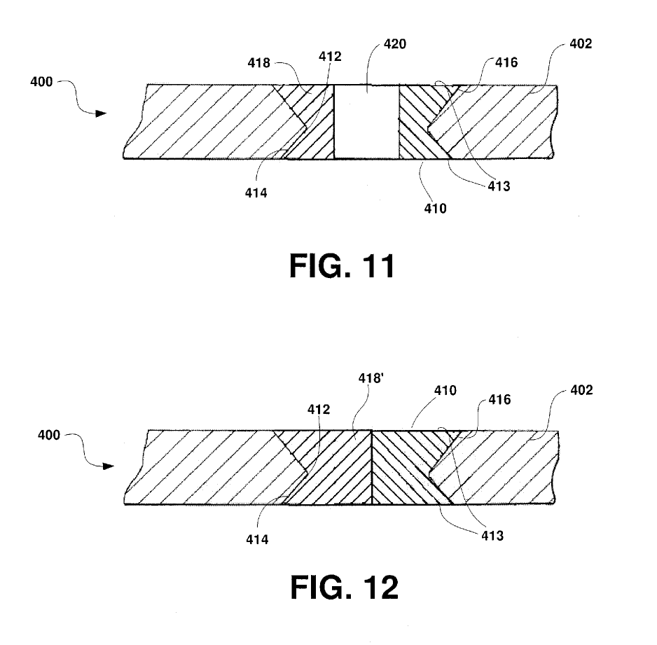

As shown in FIG. 11, a bone plate 400 according to another embodiment of the invention includes a body 402 constructed of a metal as described above in regard to the core 102 of the nail 100 to provide stiffness greater than that attainable by a strictly polymer construction. Inserts 418 are then secured within apertures 410 of the body 402 providing a plurality of locations for engaging locking elements as described above (e.g., locking elements 106 and 306). As shown in FIG. 11, the inserts 418 may include a bore 420 extending therethrough to receive a locking element. Furthermore, the polymeric inserts 418 may be shaped to prevent their becoming dislodged from the apertures 410. For example, the inserts 418 may include a reduced diameter central portion 412 between enlarged end portions 413. When inserted into a correspondingly shaped aperture 410, the enlarged end portions 413 will be too large to pass through the reduced diameter central portion of the aperture 410. As shown in the cross-section of FIG. 14, the enlarged end portions 413 comprise angular faces 414, 416 flaring outward toward the outer surfaces of the plate 400. The fixation plate 400 is implanted in substantially the same manner described above for the plate 300 except that the inserts 418 will generally be pre-placed within the apertures 410 and bonded to the plate 400 prior to the procedure.

In yet another alternate embodiment, as shown in FIG. 12, polymeric inserts 418′ can be formed in a solid configuration, wherein a bone screw may be screwed therethrough and subsequently permanently bonded thereto via ultrasonic welding.

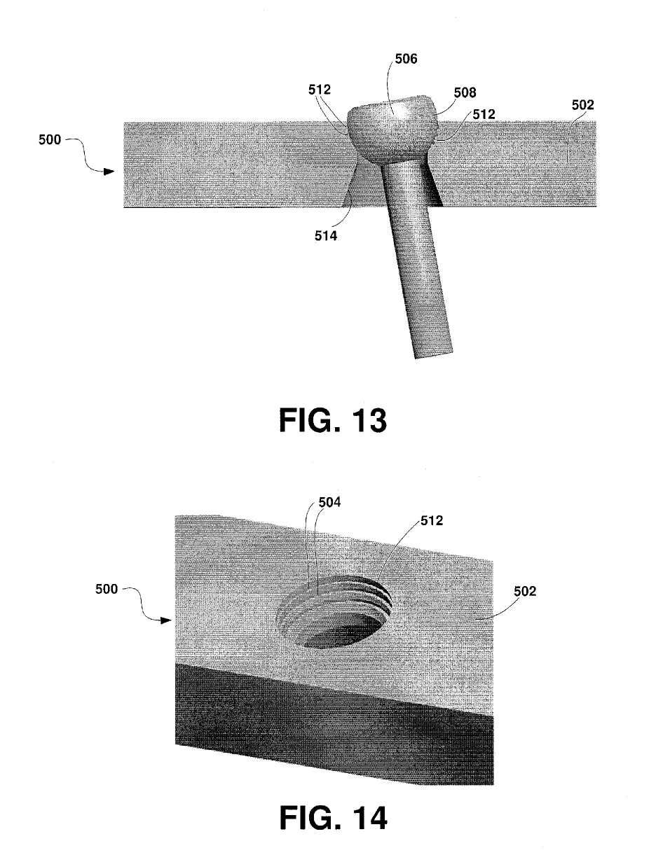

FIGS. 13 and 14 shows another alternate embodiment of the present invention, comprising a bone plate 500 with a body 502 provided with a hole 512 formed therethrough. A proximal portion 514 of the hole 512 is substantially spherically curved substantially matching a curvature of a locking element 506 adapted to be received in the hole 512. A distal portion 514 of the hole 512 tapers linearly outward from a central portion thereof. The wall of the proximal portion 514 of the hole 512 is formed with annular rings 504 machined into the material of the body 502 (e.g., a rigid material such as any of the above mentioned metals). Accordingly, when a locking element 506 is inserted through the hole 512 in accordance with the method disclosed with respect to earlier embodiments, a spherical head 508 of the locking element 506 engages the spherical proximal portion 514 and energy (e.g., ultrasonic vibration) is applied to the locking element 506 to melt the polymeric material of the head 508 into the annular rings 504. This exemplary embodiment precludes the requirement of having polymeric portions formed on the bone plate 500.

The present invention has been described with reference to specific exemplary embodiments. Those skilled in the art will understand that changes may be made in details, particularly in matters of shape, size, material and arrangement of parts. Accordingly, various modifications, combinations and changes may be made to the embodiments. The specifications and drawings are, therefore, to be regarded in an illustrative rather than a restrictive sense.

What is claimed is:

1. A device for treating bone, comprising:
a rigid body including a polymeric material extending over at least a target portion of the body to which a locking element extending into the bone is to be permanently bonded by melting a portion of an outer surface of the locking element and the polymeric material.

2. The device according to claim 1, wherein the body is formed as an intramedullary nail.

3. The device according to claim 1, wherein the locking element is formed as a locking tack with a shaft and a head having a diameter greater than that of the head.

4. The device according to claim 3, wherein the distal end of the locking tack includes at least two angled faces adapted to increase the outer surface area of the locking tack.

5. The device according to claim 1, wherein the nail includes a metallic core and a polymeric casing extending over at least target portions of the stem thereof.

6. The device according to claim 1, wherein the body is formed as a bone plate.

7. The device according to claim 6, further comprising:
a bore extending through the bone plate, the bore comprising annular rings formed along a wall thereof adapted to engage a polymeric insert received therethrough.

8. The device according to claim 7, wherein a proximal portion of the bore is formed as a half-sphere.

9. The device according to claim 6, further comprising:
a bore extending through the bone plate, the bore adapted to receive a polymeric insert therethrough.

10. The device according to claim 9, further comprising:
an opening extending through the polymeric insert for receiving the locking element.

11. The device according to claim 2, further comprising:
a non-circular end cap adapted to be received over an end of the intramedullary nail for preventing rotation of the intramedullary nail when placed in an operative configuration in the bone.

12. A method for securing an intramedullary nail, comprising:
inserting the intramedullary nail into a medullary canal of a bone until a distal tip thereof reaches a first target point along a longitudinal axis of the bone at which a locking element is to be inserted through the bone to couple to the nail, the intramedullary nail being formed with a rigid core formed of a first material including a polymeric coating extending over at least a first target portion thereof;
drilling a first locking element receiving bore through the bone to the medullary canal at the first target point;
advancing the nail further distally into the medullary canal until the distal tip is in a desired position therein;
inserting a first locking element through the first locking element receiving bore until a distal tip of the first locking element engages the target portion of the nail; and
supplying energy to the first locking element to melt a portion of the polymeric coating and the first locking element to generate a permanent bond therebetween.

13. The method of claim 12, further comprising:
cutting outlying portions of the first locking element to lie flush against an outer cortex of the bone.

14. The method of claim 12, further comprising:
drilling a second locking element receiving bore through the bone to the medullary canal at a second target point;
inserting a second locking element through the second locking element receiving bore until a distal tip of the second locking element engages the target portion of the nail; and
supplying energy to the second locking element to melt a portion of the polymeric coating and the second locking element to generate a permanent bond therebetween.

15. The method of claim 12, wherein the energy includes ultrasonic vibration.

16. The method of claim 15, wherein the ultrasonic vibration is supplied by coupling an ultrasonic generator to the first locking element.

17. The method of claim 15, further comprising:
determining that distal tip of nail has reached the first target point by fluoroscopically imaging the rigid core of the intramedullary nail.

18. The method of claim 17, wherein the rigid core is metallic.

19. The method of claim 12, wherein the first locking element includes a polymeric outer surface.

20. The method of claim 19, wherein the first locking element includes a metallic core.

21. A method for securing a bone plate, comprising:
situating a bone plate over a first target portion of a bone, the bone plate formed of a rigid body having a first opening formed therethrough, the first opening housing a polymeric portion therethrough;
inserting a first locking element through the polymeric portion and into a first locking element receiving bore formed in the first target portion of the bone; and
supplying energy to the first locking element to melt a portion of the polymeric portion and the first locking element to generate a permanent bond therebetween.

22. The method of claim 21, wherein the polymeric portion comprises a second opening formed therethrough.

23. The method of claim 21, further comprising:
screwing a bone screw through a second bore formed in the bone plate, the bone screw extending into a second target portion of the bone.

24. The method of claim 21, further comprising:
drilling a second locking element receiving bore into a second target portion of the bone;
situating the bone plate over the first and second target portions of the bone, the bone plate having a second opening formed therethrough, the second opening housing a polymeric portion therethrough, wherein the polymeric portion is situated to lie in alignment with the second locking element receiving bore;
inserting a second locking element through the polymeric portion and into the second locking element receiving bore; and
supplying energy to the second locking element to melt a portion of the polymeric portion and the second locking element to generate a permanent bond therebetween.

25. The method according to claim 21, wherein the energy includes ultrasonic vibration.
